(19) United States
(12) Patent Application Publication
(10) Pub. No.: US 2025/0235158 A1
(43) Pub. Date: Jul. 24, 2025

(54) CONTINUOUS ELECTRICAL TRACE IN INTRALUMINAL DEVICE AND ASSOCIATED DEVICES, SYSTEMS, AND METHODS

(71) Applicant: KONINKLIJKE PHILIPS N.V., EINDHOVEN (NL)

(72) Inventor: Mathijs DE WIT, ASSENDELFT (NL)

(21) Appl. No.: 18/853,938

(22) PCT Filed: Mar. 30, 2023
(86) PCT No.: PCT/EP2023/058366

§ 371 Date: Oct. 3, 2024

Related U.S. Application Data
(60) Provisional application No. 63/328,330, filed on Apr. 7, 2022.

(51) Int. Cl. A61B 5/00; A61B 5/0215; A61B 5/026; A61M 25/00; A61M 25/09

(52) U.S. Cl. CPC A61B 5/6851; A61B 5/0215; A61B 5/026; A61B 2562/227; A61M 2025/0002; A61M 2025/09175

(57) ABSTRACT

An apparatus includes an intravascular guidewire that includes a flexible elongate member configured to be positioned within a blood vessel of a patient. The flexible elongate member includes a proximal portion and a distal portion. The proximal portion includes a proximal core wire and the distal portion includes a distal core wire. The intravascular guidewire includes a sensor disposed at the distal portion of the flexible elongate member. The sensor is configured to obtain medical data related to the blood vessel while the flexible elongate member is positioned within the blood vessel. The intravascular guidewire includes a connector disposed at the proximal portion of the flexible elongate member. The intravascular guidewire includes a conductive member establishing electrical communication between the sensor and the connector. The conductive member is continuous between sensor and the connector.

### TECHNICAL FIELD

The subject matter described herein relates to intraluminal physiology sensing devices (e.g., an intravascular pressure sensing and/or flow sensing guidewire). For example, the intraluminal device may include continuous electrical traces between a distal sensor and a proximal electrical connector.

### BACKGROUND

Existing intravascular guidewires with a sensor have fine-gauge electrical wires that provide transmission of electrical signals for the sensor. Manufacture of such devices may include many steps that involve human operator or machine contact with the fine-gauge electrical wires. Because of their delicate nature, the fine-gauge electrical wires are prone to damage as a result of such contact. For example, the conductors themselves may break and the insulation may be damaged. This leads to poor or no electrical connectivity for the sensor. Additionally, the fine-gauge electrical wires typically have circular cross-section, which occupies space with the very small outer diameter of the guidewire. Further, existing intravascular guidewires sometimes require soldering to make an electrical connection between different conductors that extend along different parts of length. This introduces chance for failure at these connections, preventing electrical signals from the sensor to be received at the proximal connector.

The information included in this Background section of the specification, including any references cited herein and any description or discussion thereof, is included for technical reference purposes only and is not to be regarded as subject matter by which the scope of the disclosure is to be bound.

### SUMMARY

Disclosed are intraluminal physiology sensing devices (e.g., an intravascular pressure-sensing and/or flow-sensing guidewire) that include conductive members for power and signal communication. Example of conductive members include electrical traces formed with a conductive ink, metal disposition, electroplating, three-dimensional printed conductive polymer or metal, and/or extruded conductive material. The conductive members may be applied in a thinner layer reducing the dimensions (thickness) of the electrical connection, facilitating centering of the distal core within the wire body and removing irregularities in the surface. The sensor is electrically connected using a bonding process and additional bonding materials. The conductive members may be directly connected to the sensor or sensor mount, without the need for additional bonding process that require additional forces on the sensor. This advantageously provides a more robust electrical connection because there are no solder joints between multiple electrical conductors. This ensures that electrical signals from the sensor to be received at the proximal connector, which allows the medical professional to have intravascular pressure and/or flow information to make decisions about a patient's healthcare, which leads to improvement in health outcomes for the patient. Furthermore, the straightness, flexibility, and torque response of the intraluminal device with conductive members may be improved as well because ribbons and/or multi-filar conductor bundles may cause non-uniform storage of energy and non-uniform stiffness, resulting in local torques during use of the guidewire or catheter.

The conductive member assembly disclosed herein has particular, but not exclusive, utility for intraluminal medical catheters, guidewires, or guide catheters.

In an exemplary aspect, an apparatus is provided. The apparatus includes an intravascular guidewire comprising: a flexible elongate member configured to be positioned within a blood vessel of a patient, wherein the flexible elongate member comprises a proximal portion and a distal portion, wherein the proximal portion comprises a proximal core wire and the distal portion comprises a distal core wire; a sensor disposed at the distal portion of the flexible elongate member, wherein the sensor is configured to obtain medical data related to the blood vessel while the flexible elongate member is positioned within the blood vessel; a connector disposed at the proximal portion of the flexible elongate member; and a conductive member establishing electrical communication between the sensor and the connector, wherein the conductive member is continuous between sensor and the connector.

In some aspects, a distal portion of the conductive member is directly coupled to the sensor. In some aspects, the connector comprises a conductive band, wherein a proximal portion of the conductive member is directly coupled to the conductive band. In some aspects, the intravascular guidewire comprises plurality of conductive members establish electrical communication between the sensor and the connector, wherein each conductive member of the plurality of conductive members is continuous between the sensor and the connector. In some aspects, a distal portion of the proximal core wire is coupled to a proximal portion of the distal core wire at a location along a length of the flexible elongate member, wherein the conductive member is continuous across the location. In some aspects, the location comprises a tubular member, wherein the distal portion of the proximal core wire and the proximal portion of the distal core wire are disposed inside of the tubular member, wherein the conductive member is disposed outside of the tubular member. In some aspects, the flexible elongate member further comprises a first polymer layer disposed over the proximal core wire and the distal core wire, wherein the first polymer layer is configured to provide insulation for the conductive member from the proximal core wire and the distal core. In some aspects, the intravascular guidewire further comprises a sensor mount, wherein the sensor is coupled to the sensor mount, wherein the first polymer layer is disposed over the sensor mount. In some aspects, the first polymer layer comprises an opening exposing the sensor. In some aspects, the flexible elongate member further comprises a second polymer layer disposed over the conductive member. In some aspects, the flexible elongate member further comprises a hydrophilic coating disposed over the second polymer layer. In some aspects, in a cross-section, a perimeter of the conductive member is completely surrounded by at least one of the first polymer layer or the second polymer layer. In some aspects, the conductive member comprises an electrical trace. In some aspects, the electrical trace comprises at least one of a conductive ink, metal disposition, electroplating, three-dimensional printed conductive polymer or metal, or extruded conductive material. In some aspects, the sensor comprises at least one of a pressure sensor or a flow sensor.

In an exemplary aspect, an apparatus is provided. The apparatus includes an intravascular guidewire comprising: a flexible elongate member configured to be positioned within a blood vessel of a patient, wherein the flexible elongate member comprises a proximal portion and a distal portion, wherein the proximal portion comprises a proximal core wire and the distal portion comprises a distal core wire, wherein proximal core wire is coupled to the distal core wire at a location along a length of the flexible elongate member; at least one of a pressure sensor or a flow sensor disposed at the distal portion of the flexible elongate member, wherein at least one of the pressure sensor or the flow sensor is configured to obtain at least one of pressure data or flow data, respectively, related to the blood vessel while the flexible elongate member is positioned within the blood vessel; a connector disposed at the proximal portion of the flexible elongate member; and a conductive ink trace establishing electrical communication between the sensor and the connector, wherein the electrical trace is continuous between sensor and the connector, including across the location.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the metal ink conductor assembly, as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a diagrammatic top view of an intravascular device, according to aspects of the present disclosure.

FIG. 2 is a diagrammatic side view of an intravascular sensing system that includes an intravascular device, according to aspects of the present disclosure.

FIGS. 3A-3H are diagrammatic cross-sectional top-views of an intravascular device according to aspects of the present disclosure.

FIG. 4 is a diagrammatic view of an intravascular device according to aspects of the present disclosure.

FIG. 5 is a diagrammatic view of a sensor mount/housing coupled to a distal core of an intravascular device according to aspects of the present disclosure.

FIG. 6 is a diagrammatic view of a first polymer coating applied to an intravascular device according to aspects of the present disclosure.

FIG. 7 is a diagrammatic view of exposing a portion of a sensor mount/housing of an intravascular device according to aspects of the present disclosure.

FIG. 8 is a diagrammatic view of mounting a sensor to a sensor mount/housing of an intravascular device according to aspects of the present disclosure.

FIG. 9 is a schematic top view of a sensor of an intravascular device according to aspects of the present disclosure.

FIG. 10 is a diagrammatic view of applying one or more conductive layer along the length of an intravascular device 102 according to aspects of the present disclosure.

FIG. 11 is a diagrammatic view of one or more conductive layers coupled to one of one or more electrical contact pads and of a sensor of an intravascular device according to aspects of the present disclosure.

FIG. 12 illustrates a cross-sectional view of an intravascular device of FIG. 11, as seen along the lines of the section A-A taken therein, according to aspects of the present disclosure.

FIG. 13 is a diagrammatic view of applying a second polymer coating applied to an intravascular device according to aspects of the present disclosure.

FIG. 14 is a diagrammatic view of a second polymer coating applied to a distal core a of an intravascular device an according to aspects of the present disclosure.

FIG. 15 is a diagrammatic view of applying conductive portions to an intravascular device according to aspects of the present disclosure.

FIG. 16 illustrates a cross-sectional view of an intravascular device of FIG. 15, as seen along the lines of the section B-B taken therein, according to aspects of the present disclosure.

FIG. 17 illustrates a cross-sectional view of an intravascular device of FIG. 15, as seen along the lines of the section C-C taken therein, according to aspects of the present disclosure.

FIG. 18 illustrates a cross-sectional view of an intravascular device of FIG. 11, as seen along the lines of the section A-A taken therein, according to aspects of the present disclosure.

FIG. 19 illustrates a cross-sectional view of an intravascular device according to aspects of the present disclosure.

FIG. 20 illustrates a cross-sectional view of an intravascular device according to aspects of the present disclosure.

### DETAILED DESCRIPTION

Disclosed is a conductive member assembly that provides an improved electrical/mechanical performance for an intraluminal sensing device in an intraluminal system and that eliminates manufacturing issues associated with conductive filars. Multi-filar bundles (e.g., bifilar or trifilar) are groupings or multiple (e.g., two or three) conductors, with each conductor being surrounded by insulating coating. During the manufacturing or assembly process of the intraluminal sensing device the multi-filar bundle passes through an inside of a hypotube in a hypotube joint area of the intraluminal sensing device. The hypotube joint area includes five main elements/components: the hypotube, a proximal core, a distal core, the multi-filar bundle, and an adhesive. Assembling the hypotube joint requires significant ability and skill from the operators that put the hypotube joint together. Having a lot of components and pieces to handle/assemble increase the potential for component damage and compromised electrical signal. The multi-filar bundle is a very delicate component that is easily damaged during assembly as the multi-filar bundle needs to be fed through the inside of the hypotube.

Replacing embedded filars with conductive members that are placed on the outside of the hypotube and/or replace the hypotube altogether, enables the use of conductive materials that applied in a thinner layer reducing the dimensions (thickness) of the electrical connection, facilitating centering of the distal core within the wire body and removing irregularities in the surface. The sensor is electrically connected using a bonding process and additional bonding materials. Conductive members may be directly connected to the sensor or sensor mount, without the need for additional bonding process that require additional forces on the sensor. Because ribbons and/or multi-filar conductor bundles add stiffness and local torques to a guidewire or catheter, the straightness and torque response of the intraluminal device with conductive members may be improved as well, which may lead to a reduction in mechanical whipping responses when the device is manipulated within intravascular anatomy. In some instances, whipping may also be referred to as jump torquing. Whipping occurs when the distal portion of the guidewire does not smoothly rotate with the proximal portion of the guidewire (e.g., which is controlled by a user), instead the distal portion of the guidewire rapidly rotates inside the blood vessel, releasing the stored energy at once to catch up to the rotational position of the proximal portion of the guidewire.

Other manufacturing issues may also be alleviated by using the conductive member assembly of the present disclosure. For example, in a current manufacturing process, a proximal end of a distal core and a distal end of a proximal core are reduced in diameter so that the multi-filar bundle may fit inside of the hypotube and allow sufficient space for the multi-filar bundle. This reduction in diameter makes it more likely for this area of the wire to kink during bending/handling. By applying the conductive members to the outside of the hypotube or replace the hypotube altogether with such conductive members, the conductive member assembly reduces or eliminates these difficulties.

Example devices incorporating a multi-filar conductor bundle and/or conductive ribbons include intraluminal medical guidewire devices as described for example in U.S. Pat. No. 10,595,820 B2, U.S. Patent Publication Nos. 2014/0187874, 2016/0058977, and 2015/0273187, and in U.S. Provisional Patent Application No. 62/552,993 (filed Aug. 31, 2017), each of which is hereby incorporated by reference in its entirety as though fully set forth herein.

These descriptions are provided for exemplary purposes only and should not be considered to limit the scope of the metal ink conductor assembly. Certain features may be added, removed, or modified without departing from the spirit of the claimed subject matter.

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one aspect may be combined with the features, components, and/or steps described with respect to other aspects of the present disclosure. Further, while the aspects of the present disclosure may be described with respect to a blood vessel, it will be understood that the devices, systems, and methods described herein may be configured for use in any suitable anatomical structure or body lumen including a blood vessel, blood vessel lumen, an esophagus, eustachian tube, urethra, fallopian tube, intestine, colon, and/or any other suitable anatomical structure or body lumen. In other aspects, the devices, systems, and methods described herein may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood vessels, chambers or other parts of the heart, and/or other systems of the body. In addition to natural structures, the device may be used to examine man-made structures such as, but without limitation, heart valves, stents, shunts, filters, and other devices. For the sake of brevity, however, the numerous iterations of these combinations will not be described separately.

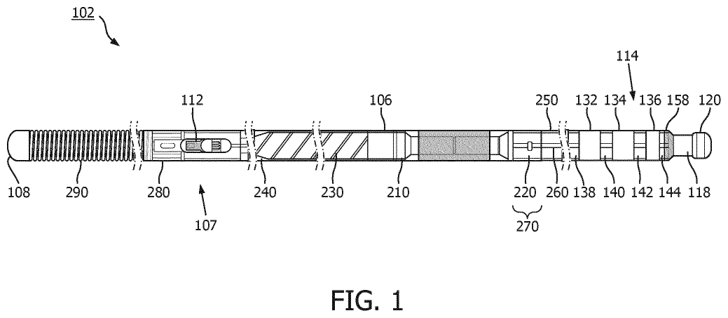

FIG. 1 is a diagrammatic top view of an intravascular device 102, according to aspects of the present disclosure. The intravascular device 102 may be an intravascular, intraluminal, or endoluminal device, such as a guidewire, a catheter, or a guide catheter sized and shaped for positioning within a blood vessel of a patient. The intravascular device 102 may include a sensor 112. For example, the sensor 112 may be a pressure sensor configured to measure a pressure of blood flow within the vessel of the patient. The intravascular device 102 includes the flexible elongate member 106. The sensor 112 is disposed at the distal portion 107, also referred to as a distal subassembly, of the flexible elongate member 106. The sensor 112 may be mounted at the distal portion 107 within a housing 280 in some aspects. A flexible tip coil 290 extends between the housing 280 and the distal end 108. The connection portion 114 is disposed at the proximal portion 109, also referred to as a proximal subassembly, of the flexible elongate member 106. The connection portion includes the conductive portions 132, 134, 136. In some aspects, the conductive portions 132, 134, 136 may be conductive ink that is printed and/or deposited around the flexible elongate member 106. In some aspects, the conductive portions 132, 134, 136 may be conductive, metallic rings that are positioned around the flexible elongate member. The locking section 118 and knob or retention section 120 are disposed at the proximal portion 109 of the flexible elongate member 106.

The intravascular device 102 in FIG. 1 includes a distal core 210 and a proximal core 220. The distal core 210 and the proximal core 220 are metallic components forming part of the body of the intravascular device 102. For example, the distal core 210 and the proximal core 220 are flexible metallic rods that provide structure for the flexible elongate member 106. The diameter of the distal core 210 and the proximal core 220 that electrically and mechanically couples the distal core 210 to the proximal core 220 may vary along its length. A joint between the distal core 210 and proximal core 220, which electrically and mechanically couples the distal core 210 to the proximal core 220, is surrounded and contained by a hypotube 215, which is a tubular member.

In some aspects, the intravascular device 102 includes a distal assembly and a proximal assembly that are electrically and mechanically joined together, which results in electrical communication between the sensor 112 and the conductive portions 132, 134, 136. For example, pressure data obtained by the sensor 112 (in this example, sensor 112 is a pressure sensor) may be transmitted to the conductive portions 132, 134, 136. Control signals from a computer in communication with the intravascular device 102 may be transmitted to the sensor 112 via the conductive portions 132, 134, 136. The distal subassembly may include the distal core 210. The distal subassembly may also include the sensor 112, conductive members 230, and/or one or more layers of insulative polymer/plastic 240 surrounding the conductive members 230 and the core 210. For example, the polymer/plastic layer(s) may protect the conductive members 230. The proximal subassembly may include the proximal core 220. The proximal subassembly may also include one or more layers of polymer layer(s) 250 (hereinafter polymer layer 250) surrounding the proximal core 220 and/or conductive ribbons 260 embedded within the one or more layers of polymer layer(s) 250. In some aspects, the proximal subassembly and the distal subassembly may be separately manufactured. During the assembly process for the intravascular device 102, the proximal subassembly and the distal subassembly may be electrically and mechanically joined together. As used herein, flexible elongate member may refer to one or more components along the entire length of the intravascular device 102, one or more components of the proximal subassembly (e.g., including the proximal core 220, etc.), and/or one or more components the distal subassembly (e.g., including the distal core 210, etc.).

In various aspects, the intravascular device 102 may include one, two, three, or more core wires, also referred to as core members, extending along its length. For example, a single core wire may extend substantially along the entire length of the flexible elongate member 106. In such aspects, the locking section 118 and the knob or retention section 120 may be integrally formed at the proximal portion of the single core wire. The sensor 112 may be secured at the distal portion of the single core wire. In other aspects, such as the illustration in FIG. 1, the locking section 118 and the knob or retention section 120 may be integrally formed at the proximal portion of the proximal core 220. The sensor 112 may be secured at the distal portion of the distal core 210. The intravascular device 102 includes one or more conductive members 230 in communication with the sensor 112. For example, the conductive members 230 may be one or more electrical wires that are directly in communication with the sensor 112. In some instances, the conductive members 230 are electrically and mechanically coupled to and in electrical communication with the sensor 112 by, e.g., soldering. In some instances, the conductive members 230 include two or three electrical wires (e.g., a bifilar cable or a trifilar cable). An individual electrical wire may include a bare metallic conductor surrounded by one or more insulating layers. The conductive members 230 may extend along the length of the distal core 210. For example, at least a portion of the conductive members 230 may be spirally wrapped around the distal core 210.

The intravascular device 102 includes one or more conductive ribbons 260 at the proximal portion of the flexible elongate member 106. The conductive ribbons 260 are embedded within polymer layer(s) 250. The conductive ribbons 260 are directly in communication with the conductive portions 132, 134, and/or 136. In some instances, the conductive members 230 are electrically and mechanically coupled to and in electrical communication with the sensor 112 by, e.g., soldering. In some instances, the conductive portions 132, 134, and/or 136 include conductive ink (e.g., metallic nano-ink, such as silver or gold nano-ink) that is deposited or printed directed over the conductive ribbons 260.

As described herein, electrical communication between the conductive members 230 and the conductive ribbons 260 may be established at the connection region 270 of the flexible elongate member 106. By establishing electrical communication between the conductive members 230 and the conductive ribbons 260, the conductive portions 132, 134, 136 may be in electrically communication with the sensor 112.

In some aspects represented by FIG. 1, intravascular device 102 includes the locking section 118 and the knob or retention section 120. To form the locking section 118, a machining process is necessary to remove the polymer layer 250 and the conductive ribbons 260 in the locking section 118 and to shape proximal core 220 in the locking section 118 to the desired shape. As shown in FIG. 1, the locking section 118 includes a reduced diameter while the knob or retention section 120 has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons.

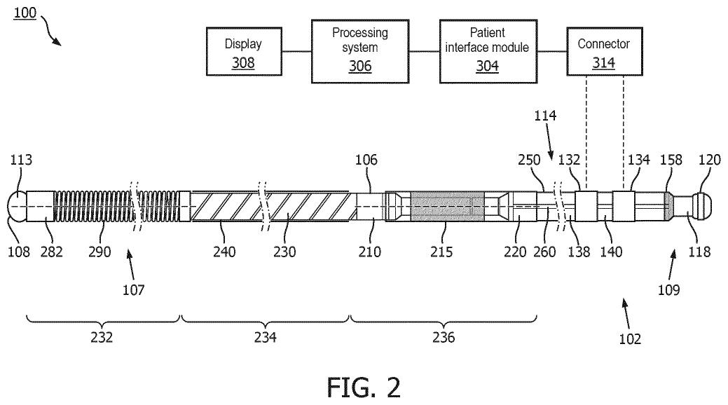

FIG. 2 is a diagrammatic side view of an intraluminal (e.g., intravascular) sensing system 100 that includes an intravascular device 102 includes conductive members 230 (e.g., a multi-filar electrical conductor bundle) and conductive ribbons 260, according to aspects of the present disclosure. The intravascular device 102 may be an intravascular guidewire sized and shaped for positioning within a blood vessel of a patient. The intravascular device 102 includes a distal end 108 and a sensor 113. For example, the sensor 113 may be a pressure sensor and/or flow sensor configured to measure a pressure of blood flow within the vessel of the patient, or another type of sensor including but not limited to a temperature or imaging sensor, or combination sensor measuring more than one property. For example, the flow data obtained by a flow sensor may be used to calculate physiological variables such as coronary flow reserve (CFR). The intravascular device 102 includes a flexible elongate member 106. The sensor 113 is disposed at a distal portion 107 of the flexible elongate member 106. The sensor 113 may be mounted at the distal portion 107 within a housing 282 in some aspects. A flexible tip coil 290 extends distally from the housing 282 at the distal portion 107 of the flexible elongate member 106. A connection portion 114 located at a proximal end of the flexible elongate member 106 includes conductive portions 132, 134. In some aspects, the conductive portions 132, 134 may be conductive ink that is printed and/or deposited around the connection portion 114 of the flexible elongate member 106. In some aspects, the conductive portions 132, 134 are conductive, may be metallic bands or rings that are positioned around the flexible elongate member. A locking area is formed by a collar or locking section 118 and knob or retention section 120 are disposed at the proximal portion 109 of the flexible elongate member 106.

The intravascular device 102 in FIG. 2 includes core wire including a distal core 210 and a proximal core 220. In some instances, the distal core 210 and the proximal core 220 are metallic components forming part of the body of the intravascular device 102. For example, the distal core 210 and the proximal core 220 may be flexible metallic rods that provide structure for the flexible elongate member 106. The distal core 210 and/or the proximal core 220 may be made of a metal or metal alloy. For example, the distal core 210 and/or the proximal core 220 may be made of stainless steel, Nitinol, Titanium, nickel-cobalt-chromium-molybdenum alloy (e.g., MP35N), and/or other suitable materials. In some instances, the distal core 210 and/or the proximal core 220 may be made from a stiff graphite or similar composite material. In some aspects, the distal core 210 and the proximal core 220 are made of the same material. In other aspects, the distal core 210 and the proximal core 220 are made of different materials. The diameter of the distal core 210 and the proximal core 220 may vary along their respective lengths. A joint between the distal core 210 and proximal core 220 is surrounded and contained by a hypotube 215. The sensor 113 may in some cases be positioned at a distal end of the distal core 210.

In some aspects, the intravascular device 102 includes a distal subassembly and a proximal subassembly that are electrically and mechanically joined together, which creates an electrical communication between the sensor 113 and the conductive portions 132, 134. For example, flow data obtained by the sensor 113 (in this example, sensor 113 is a flow sensor) may be transmitted to the conductive portions 132, 134. In an exemplary aspect, the sensor 113 is a single ultrasound transducer element. The transducer element emits ultrasound signals and receives echoes. The transducer element generates electrical signals representative of the echoes. The signal carrying filars carry this electrical signal from the sensor at the distal portion to the connector at the proximal portion. The processing system 306 processes the electrical signals to extract the flow velocity of the fluid.

Control signals from the processing system 306 (e.g., a processor circuit of the processing system 306) in communication with the intravascular device 102 may be transmitted to the sensor 113 via a connector 314 that is attached to the conductive portions 132, 134. The distal subassembly may include the distal core 210. The distal subassembly may also include the sensor 113, the conductive members 230, and/or one or more layers of insulative polymer/plastic 240 surrounding the conductive members 230 and the distal core 210. For example, the polymer/plastic layer(s) may insulate and protect the conductive members of the conductive members 230. The proximal subassembly may include the proximal core 220. The proximal subassembly may also include one or more polymer layers 250 (hereinafter polymer layer 250) surrounding the proximal core 220 and/or conductive ribbons 260 embedded within the one or more insulative and/or polymer layer 250. In some aspects, the proximal subassembly and the distal subassembly are separately manufactured. During the assembly process for the intravascular device 102, the proximal subassembly and the distal subassembly may be electrically and mechanically joined together. As used herein, flexible elongate member may refer to one or more components along the entire length of the intravascular device 102, one or more components of the proximal subassembly (e.g., including the proximal core 220, etc.), and/or one or more components the distal subassembly (e.g., including the distal core 210, etc.). Accordingly, flexible elongate member may refer to the combined proximal and distal subassemblies described above. The joint between the proximal core 220 and distal core 210 is surrounded by the hypotube 215, which is a tubular member.

In various aspects, the intravascular device 102 may include one, two, three, or more core wires extending along its length. For example, a single core wire may extend substantially along the entire length of the flexible elongate member 106. In such aspects, the locking section 118 and the knob or retention section 120 may be integrally formed at the proximal portion of the single core wire. The sensor 113 may be secured at the distal portion of the single core wire. In other aspects, such as the illustration in FIG. 2, the locking section 118 and the knob or retention section 120 may be integrally formed at the proximal portion of the proximal core 220. The sensor 113 may be secured at the distal portion of the distal core 210. The intravascular device 102 includes one or more conductive members 230 (e.g., a multi-filar conductor bundle or cable) in communication with the sensor 113. For example, the conductive members 230 may be one or more electrical wires that are directly in communication with the sensor 113. In some instances, the conductive members 230 are electrically and mechanically coupled to and in electrical communication with the sensor 113 by, e.g., soldering. In some instances, the conductive members 230 includes two or three electrical wires (e.g., a bifilar cable or a trifilar cable). An individual electrical wire may include a bare metallic conductor surrounded by one or more insulating layers. The conductive members 230 may extend along the length of the distal core 210. For example, at least a portion of the conductive members 230 may be spirally wrapped around the distal core 210, minimizing or eliminating whipping of the distal core within tortuous anatomy.

The intravascular device 102 includes one or more conductive ribbons 260 at the proximal portion of the flexible elongate member 106. The conductive ribbons 260 are embedded within polymer layer 250. The conductive ribbons 260 are directly in communication with the conductive portions 132 and/or 134. In some instances, conductive members 230 are electrically and mechanically coupled to and in electrical communication with the sensor 113 by, e.g., soldering. In some instances, the conductive portions 132 and/or 134 includes conductive ink (e.g., metallic nano-ink, such as copper, silver, gold, or aluminum nano-ink) that is deposited or printed directed over the conductive ribbons 260.

As described herein, electrical communication between the conductive members 230 and the conductive ribbons 260 may be established at the connection portion 114 of the flexible elongate member 106. By establishing electrical communication between the conductive members 230 and the conductive ribbons 260, the conductive portions 132, 134 may be in electrical communication with the sensor 113.

In some aspects represented by FIG. 1, the intravascular device 102 includes a locking section 118 and knob or retention section 120. To form locking section 118, a machining process is used to remove the polymer layer 250 and conductive ribbons 260 in locking section 118 and to shape proximal core 220 in locking section 118 to the desired shape. As shown in FIG. 1, locking section 118 includes a reduced diameter while knob or retention has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons 260.

In some aspects, a connector 314 provides electrical connectivity between the conductive portions 132, 134 and a patient interface monitor 304. The Patient Interface Monitor (PIM) 304 may in some cases connect to a console or processing system 306, which includes or is in communication with a display 308.

The intraluminal sensing system 100 may be deployed in a catheterization laboratory having a control room. The processing system 306 may be located in the control room. Optionally, the processing system 306 may be located elsewhere, such as in the catheterization laboratory itself. The catheterization laboratory may include a sterile field while its associated control room may or may not be sterile depending on the procedure to be performed and/or on the health care facility. In some aspects, the intravascular device 102 may be controlled from a remote location such as the control room, such that an operator is not required to be in close proximity to the patient.

The intravascular device 102, PIM 304, and display 308 may be communicatively coupled directly or indirectly to the processing system 306. These elements may be communicatively coupled to the processing system 306 via a wired connection such as the conductive members 230, which is a standard copper multi-filar conductor bundle. The processing system 306 may be communicatively coupled to one or more data networks, e.g., a TCP/IP-based local area network (LAN). In other aspects, different protocols may be utilized such as Synchronous Optical Networking (SONET). In some cases, the processing system 306 may be communicatively coupled to a wide area network (WAN).

The PIM 304 transfers the received signals to the processing system 306 where the information is processed and displayed (e.g., as physiology data in graphical, symbolic, or alphanumeric form) on the display 308. The console or processing system 306 may include a processor and a memory. The processing system 306 may be operable to facilitate the features of the intraluminal sensing system 100 described herein. For example, the processor may execute computer readable instructions stored on the non-transitory tangible computer readable medium.

The PIM 304 facilitates communication of signals between the processing system 306 and the intravascular device 102. The PIM 304 may be communicatively positioned between the processing system 306 and the intravascular device 102. In some aspects, the PIM 304 performs preliminary processing of data prior to relaying the data to the processing system 306. In examples of such aspects, the PIM 304 performs amplification, filtering, and/or aggregating of the data. In an aspect, the PIM 304 also supplies high- and low-voltage DC power to support operation of the intravascular device 102 via the conductive members 230.

A multi-filar cable or transmission line bundle, such as conductive members 230, may include a plurality of conductors, including one, two, three, four, five, six, seven, or more conductors. In the example shown in FIG. 2, the conductive members 230 includes two straight portions 232 and 236, where the conductive members 230 lies parallel to a longitudinal axis of the flexible elongate member 106, and a spiral portion 234, where the conductive members 230 is wrapped around the exterior of the flexible elongate member 106 and then overcoated with an insulative polymer/plastic 240. Communication, if any, along the conductive members 230 may be through numerous methods or protocols, including serial, parallel, and otherwise, where one or more filars of the conductive members 230 carry signals. One or more filars of the conductive members 230 may also carry direct current (DC) power, alternating current (AC) power, or serve as a ground connection.

The display or monitor 308 may be a display device such as a computer monitor or other type of screen. The display or monitor 308 may be used to display selectable prompts, instructions, and visualizations of imaging data to a user. In some aspects, the display 308 may be used to provide a procedure-specific workflow to a user to complete an intraluminal imaging procedure.

Before continuing, it should be noted that the examples described above are provided for purposes of illustration and are not intended to be limiting. Other devices and/or device configurations may be utilized to carry out the operations described herein.

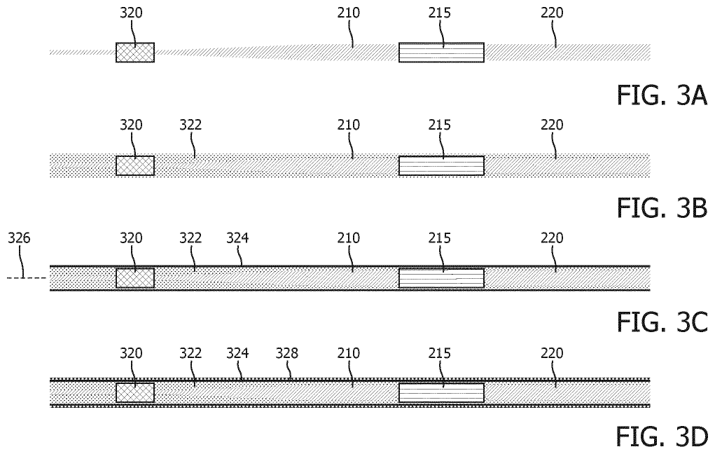

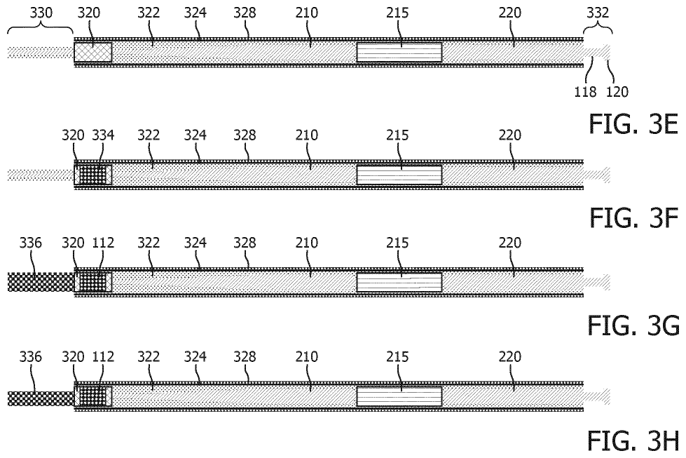

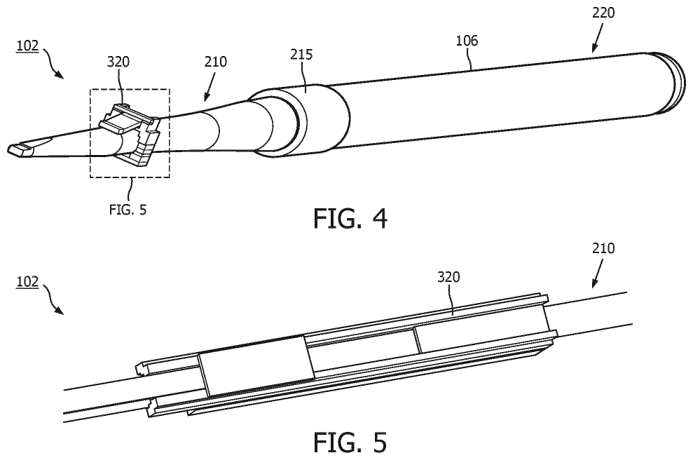

FIGS. 3A-3H are diagrammatic cross-sectional top-views of an intravascular device 102 according to aspects of the present disclosure. In FIG. 3A, visible are a proximal core 220 and a distal core 210. In some instances, the proximal core 220 and the distal core 210 may be a single core wire. In some instances, the proximal core 220 and the distal core 210 are separate core wires. In some aspects, the distal core 210 and the proximal core 220 are metallic components forming part of the body of the intravascular device 102. For example, the distal core 210 and the proximal core 220 are flexible metallic rods that provide structure for the flexible elongate member 106. In some instances, the distal core 210 and the proximal core 220 are electrically conductive. In some instances, the distal core 210 is electrically and mechanically coupled to the proximal core 220. That is, in some instances, at a location along a length of the flexible elongate member 106, a distal portion of the proximal core 220 is coupled to a proximal portion of the distal core 210. In some instance, the location may be multiple locations along the length of the flexible elongate member 106. In some instances, one of the multiple locations may be a joint between the distal core 210 and proximal core 220, which electrically and mechanically couples the distal core 210 to the proximal core 220, is surrounded and contained by a hypotube 215, which is a tubular member. In some instances, a diameter of the distal core 210 and the proximal core 220 may vary along its length. A sensor mount/housing 320 is coupled to the distal core 210 at a length from a distal end of the distal core 210 (see, e.g., FIGS. 4 and 5).

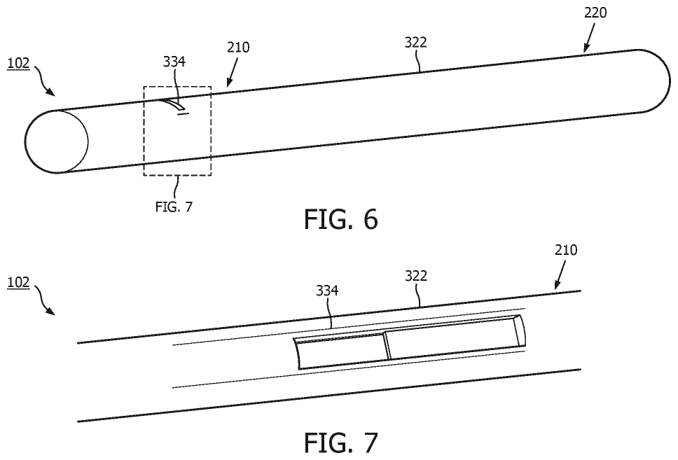

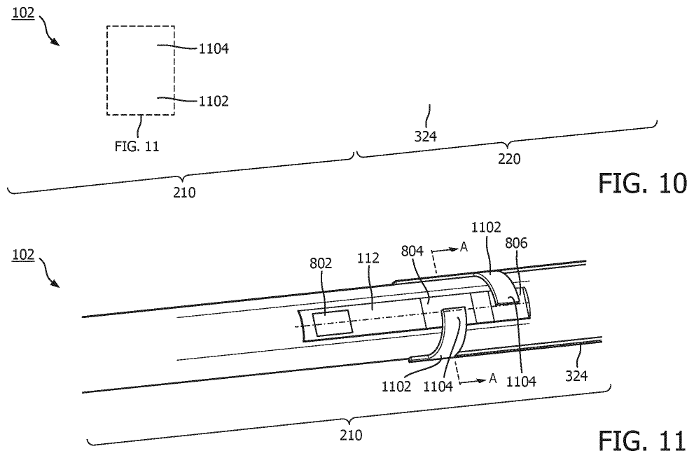

In the aspects of FIG. 3B, a first polymer coating 322 may be applied along a length of the intravascular device 102 from a proximal end of the proximal core 220 to a distal end of the distal core 210 surrounding the proximal core 220 and the distal core 210 (see, e.g., FIG. 6). In that regard, the first polymer layer 322 is disposed over, e.g., in direct contact and/or partially/fully surround in cross-section, the proximal core 220 and the distal core 210. The first polymer coating 322 may be an insulating material that is disposed between the core wire and one or more conductive layers 324. In the aspects of FIG. 3C, the one or more conductive layers 324 may then be applied along the length of the intravascular device 102 on top of the first polymer coating 322 from the proximal end of the proximal core 220 to the distal end of the distal core 210 (see, e.g., FIGS. 10, 11, and 12). In some aspects, the one or more conductive layers 324 may be, for example, an electrical trace, conductive ink, metal disposition, electroplating, three-dimensional printed conductive polymer or metal, extruded conductive material, etc. In some aspects, the one or more conductive layers 324 are positioned over the hypotube 215 that joins the proximal core 220 to the distal core 210, such that the one or more conductive layers 324 is disposed outside of the hypotube 215, i.e. the tubular member. In some aspects, the one or more conductive layers 324 is different than the proximal core 220 and the distal core 210. For example, the proximal core 220 and the distal core 210 provide structure for the intravascular device 102. The proximal core 220 and the distal core 210 provide different mechanical properties for the intravascular device 102. The one or more conductive layers 324 provide electrical communication. While in some aspects, the proximal core 220 and the distal core 210 also provide electrical communication, with two core wires, the proximal core 220 and the distal core 210 electrical pathway is not continuous between the sensor and the conductive portions because of the interconnection between the proximal core 220 and the distal core 210. The one or more conductive layers 324 are continuous length, uninterrupted, free from electrical interconnection, such that an individual (e.g., each single) conductive member extends continuously and uninterrupted. In some instances, the one or more conductive layers 324 may include one, two, three, four, five, six, seven, or more conductor layers. In some aspects, the one or more conductive layers 324 are formed parallel to a longitudinal axis 326. In some aspects, the one or more conductive layers 324 are uniformly separated from each other. In some aspects, the one or more conductive layers 324 are separated from each other by a predetermined space. For example, in some instances embodiment, 5 μm conductive layers 324 are separated by 5 μm of space. In some aspects, the one or more conductive layers 324 are separated by a uniform distance from the sensor mount/housing 320.

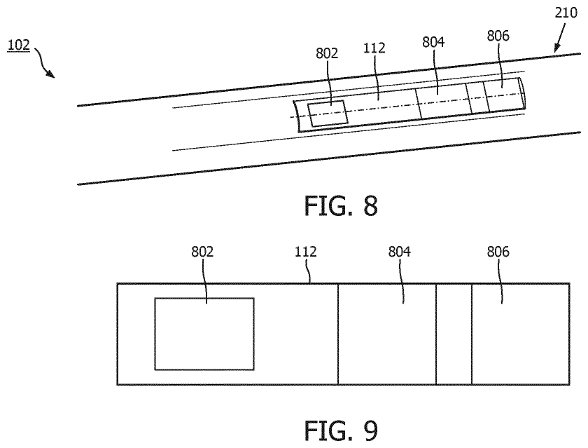

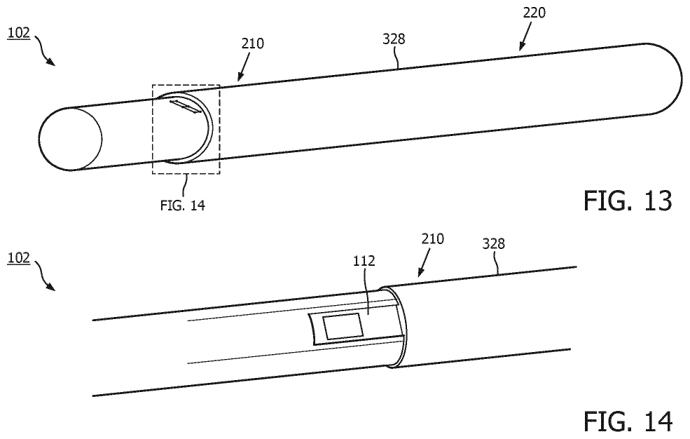

In the aspects of FIG. 3D, a second polymer coating 328 is applied along a length of the intravascular device 102 from a proximal end of the proximal core 220 to a distal end of the distal core 210 surrounding exposed portions of the proximal core 220 and the distal core 210 where the one or more conductive layers 324 are not formed and the portions where the one or more conductive layers 324 are formed (see, e.g., FIG. 13). Thus, in a cross-section, a perimeter of the one or more conductive layers 324 is completely surrounded by at least one of the first polymer coating 322 or the second polymer coating 328. In the aspects of FIG. 3E, a portion 330 of the first polymer coating 322, the one or more conductive layers 324, and/or the second polymer coating 328 on the distal end of the distal core 210 is ablated and/or ground to receive a tip coil. In the aspects of FIG. 3E, a portion 332 of the first polymer coating 322, the one or more conductive layers 324, and/or the second polymer coating 328 on the proximal end of the proximal core 220 is ablated and/or ground to form the locking section 118 and the knob or retention section 120. In the aspects of FIG. 3F, a portion 334 of the first polymer coating 322, the one or more conductive layers 324, and/or the second polymer coating 328 is ablated and/or ground to expose a portion, i.e., an opening, window, etc., of the sensor mount/housing 320 to form a sensor shape so that a sensor 112 may be coupled to the sensor mount/housing 320 (see, e.g., FIG. 7). In the aspects of FIG. 3G, a tip coil 336 may be coupled to the intravascular device 102 at the distal end of the distal core 210. In the aspects of FIG. 3G, a sensor 112 may be coupled to the sensor mount/housing 320 at the portion 334 (see, e.g., FIGS. 8 and 9). In some instances, the sensor 112 may be a flow sensor or a pressure sensor. In some instances, the sensor 112 may be a piezoresistive sensor or a piezoelectric sensor. In the aspects of FIG. 3H, a hydrophilic coating 340 is applied to all or some portions of the second polymer coating 328 and/or the sensor 112. In some aspects, the hydrophilic coating 340 may not be applied to the sensor 112, i.e. at the distal portion, the hydrophilic coating 340 starts just proximal of the sensor 112. In some aspects, the hydrophilic coating 340 may not be applied to the proximal connector, i.e. at the proximal portion, the hydrophilic coating 340 starts just distal of the conductive portions 132.

FIG. 4 is a diagrammatic view of an intravascular device 102 according to aspects of the present disclosure. In FIG. 4, visible are a proximal core 220 and a distal core 210. In some instances, the distal core 210 is electrically and mechanically coupled to the proximal core 220. That is, in some instances, at a location along a length of the flexible elongate member 106, a distal portion of the proximal core 220 is coupled to a proximal portion of the distal core 210. In some instance, the location may be multiple locations along the length of the flexible elongate member 106. In some instances, one of the multiple locations may be a joint between the distal core 210 and proximal core 220, which electrically and mechanically couples the distal core 210 to the proximal core 220, is surrounded and contained by a hypotube 215, which is a tubular member. As illustrated, in some instances, a diameter of the distal core 210 may vary along its length. A sensor mount/housing 320 is coupled to a distal end of the distal core 210, which is illustrated in an expanded view in FIG. 5. FIG. 5 is a diagrammatic view of a sensor mount/housing 320 coupled to a distal core 210 of an intravascular device 102 according to aspects of the present disclosure. In some aspects, the sensor mount/housing 320 coupled to the distal core 210 at a length from a distal end of the distal core 210. In some instances, the sensor mount/housing 320 surrounds the entire diameter of the distal core 210. In some instances, the sensor mount/housing 320 surrounds only a portion of the diameter of the distal core 210, such as three quarters of the diameter.

FIG. 6 is a diagrammatic view of a first polymer coating 322 applied to an intravascular device 102 according to aspects of the present disclosure. In some aspects, the first polymer coating 322 may be applied along a length of the intravascular device 102 from a proximal end of the proximal core 220 to a distal end of the distal core 210 surrounding the proximal core 220 and the distal core 210. In that regard, the first polymer layer 322 is disposed over, e.g., in direct contact and/or partially/fully surround in cross-section, the proximal core 220 and the distal core 210. In some instances, a portion 334 of the first polymer coating 322 is ablated and/or ground to form a sensor shape so that a sensor 112 may be coupled to the sensor mount/housing 320, which is illustrated in an expanded view in FIG. 7. The portion 334 may be movable and/or referenced as an opening or window. FIG. 7 is a diagrammatic view of exposing a portion of a sensor mount/housing 320 of an intravascular device 102 according to aspects of the present disclosure. In some aspects, a portion 334 of the first polymer coating 322 is ablated and/or ground to form a sensor shape so that a sensor 112 may be coupled to the sensor mount/housing 320.

FIG. 8 is a diagrammatic view of mounting a sensor 112 to a sensor mount/housing 320 of an intravascular device 102 according to aspects of the present disclosure. In some instances, the sensor 112 may be coupled such that a distal end of the sensor 112 that comprises an active sensing element 802 is positioned toward the distal end of the distal core 210 and a proximal end of the sensor 112 that comprises the electrical contact pads 804 and 806 for the sensor 112 is positioned toward a proximal end of the distal core 210. The active sensing element 802 may be movable and/or referenced as a diaphragm or membrane. The sensor 112 may be coupled to the sensor mount/housing 320 by, e.g., soldering, ultrasonic welding, conductive adhesive, etc. FIG. 9 is a schematic top view of a sensor 112 of an intravascular device 102 according to aspects of the present disclosure. In some instances, sensor 112 includes an active sensing element 802 positioned toward the distal end of the sensor 112 and one or more electrical contact pads 804 and 806 at a proximal end of the sensor 112.

FIG. 10 is a diagrammatic view of applying one or more conductive layers 324 along the length of an intravascular device 102 according to aspects of the present disclosure. In some instances, one or more conductive layers 324 may then be applied along the length of the intravascular device 102 on top of the first polymer coating 322 from the proximal end of the proximal core 220 to the distal end of the distal core 210 (FIG. 4). In some instances, the one or more conductive layers 324 may partially wrap around a diameter of the distal core 210 such that a first end 1102 of the partial wrap of the one or more conductive layers 324 couples to the one or more conductive layers 324 applied along the length of the intravascular device 102 and a second end 1104 of the partial wrap couples to one of the one or more electrical contact pads 804 and 806 of the sensor 112, which is illustrated in an expanded view in FIG. 11. Thus, in some instances, the one or more conductive layers 324 includes a longitudinal segment that extends along the length and a circumferential segment that extends circumferentially from the longitudinal segment to the sensor. In some instances, these segments may be considered to all form a continuous/uninterrupted length because the segments are all conductive members-so when the segments are applied, the segments become part of one another, so that the segments all form the same whole (e.g., no solder, ultrasonic welding, conductive adhesive, or other third components, establishes electrical communication between the segments.) The material of the segments themselves establish the electrical communication. In some aspects, the longitudinal segment itself has no interconnection/interruption.

In some aspects, the one or more conductive layers 324 may be, for example, an electrical trace, conductive ink, metal disposition, electroplating, three-dimensional printed conductive polymer or metal, extruded conductive material etc. In some aspects, the one or more conductive layers 324 are positioned over the hypotube 215 that joins the proximal core 220 to the distal core 210, such that the one or more conductive layers 324 is disposed outside of the hypotube 215, i.e. the tubular member. In some aspects, the one or more conductive layers 324 is different than the proximal core 220 and the distal core 210. For example, the proximal core 220 and the distal core 210 provide structure for the intravascular device 102. The proximal core 220 and the distal core 210 provide different mechanical properties for the intravascular device 102. The one or more conductive layers 324 provide electrical communication. While in some aspects, the proximal core 220 and the distal core 210 also provide electrical communication, with two core wires, the proximal core 220 and the distal core 210 electrical pathway is not continuous between the sensor 112 and the conductive portions 132, 134, 136 because of the interconnection between the proximal core 220 and the distal core 210. The one or more conductive layers 324 are continuous length, uninterrupted, free from electrical interconnection, such that an individual, single conductive member extends continuously and uninterrupted.

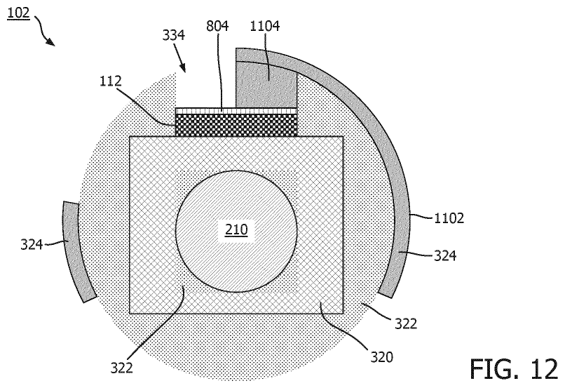

FIG. 11 is a diagrammatic view of one or more conductive layers 324 coupled to one of one or more electrical contact pads 804 and 806 of a sensor 112 of an intravascular device 102 according to aspects of the present disclosure. In some instances, a first end 1102 of the partial wrap of the one or more conductive layers 324 coupled to the one or more conductive layers 324 applied along the length of the intravascular device 102. In some instances, the second end 1104 of the partial wrap of the of the one or more conductive layers 324 may couple to one of the one or more electrical contact pads 804 and 806 of sensor 112 by, e.g., soldering, ultrasonic welding, conductive adhesive, etc. FIG. 12 illustrates a cross-sectional view of an intravascular device 102 of FIG. 11, as seen along the lines of the section A-A taken therein, according to aspects of the present disclosure. In the aspect of FIG. 12, the sensor mount/housing 320 is coupled to the distal end of the distal core 210. The first polymer coating 322 may then be applied to the distal core 210 and the sensor mount/housing 320 surrounding the distal core 210 and the sensor mount/housing 320 and filling any gaps between the distal core 210 and the sensor mount/housing 320. The portion 334 of the first polymer coating 322 is ablated and/or ground to form a sensor shape so that a sensor 112 may be coupled to the sensor mount/housing 320. One or more conductive layers 324 may then be applied to the intravascular device 102 on top of the first polymer coating 322. In some instance, the one or more conductive layers 324 partially wrap around a diameter of the distal core 210 such that a first end 1102 of the partial wrap of the one or more conductive layers 324 couples to the one or more conductive layers 324 applied along the length of the intravascular device 102 and a second end 1104 of the partial wrap couples to electrical contact pad 804 of the sensor 112.

FIG. 13 is a diagrammatic view of applying a second polymer coating 328 applied to an intravascular device 102 according to aspects of the present disclosure. In some aspects, the second polymer coating 328 may be applied along a length of the intravascular device 102 from a proximal end of the proximal core 220 to a distal end of the distal core 210 surrounding the proximal core 220 and the distal core 210. Thus, in a cross-section, a perimeter of the one or more conductive layers 324 is completely surrounded by at least one of the first polymer coating 322 or the second polymer coating 328. In some instance, the second polymer coating 328 may be applied such that the sensor 112 remains exposed, which is illustrated in an expanded view in FIG. 14. FIG. 14 is a diagrammatic view of a second polymer coating 328 applied to a distal core 210 of an intravascular device 102 according to aspects of the present disclosure. In some instances, as noted with regard to FIG. 13, the second polymer coating 328 may be applied such that the sensor 112 remains exposed. In some instances, a hydrophilic coating 340 may be applied over all or some portion of the length of the second polymer coating 328. In some aspects, the hydrophilic coating 340 may not be applied to the sensor 112, i.e. at the distal portion, the hydrophilic coating 340 starts just proximal of the sensor 112.

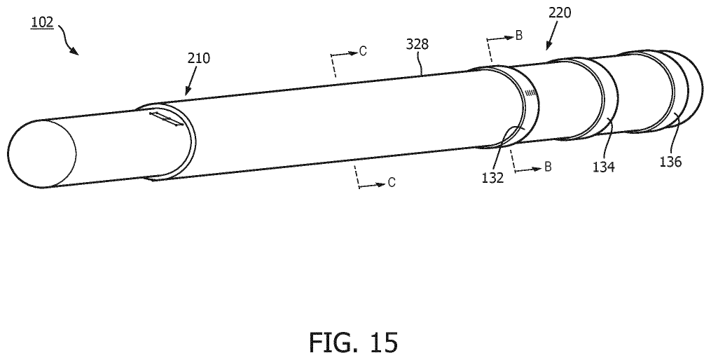

FIG. 15 is a diagrammatic view of applying conductive portions 132, 134, 136 to an intravascular device 102 according to aspects of the present disclosure. In some aspects, one or more conductive portions 132, 134, 136 are applied to a proximal end of the proximal core 220. In some aspects, the conductive portions 132, 134, 136 may be conductive ink that is printed and/or deposited around the flexible elongate member 106. In some aspects, the conductive portions 132, 134, 136 may be conductive, metallic rings that are positioned around the flexible elongate member. In some instances, the conductive portions 132, 134, 136 are formed directly over the second polymer coating 328. In some instances, each of the one or more conductive layers 324 are respectively and directly in communication with one of the conductive portions 132, 134, and/or 136. In some instances, the one or more conductive layers 324 are electrically and mechanically coupled to and in electrical communication with the sensor 112 by, e.g., soldering, ultrasonic welding, conductive adhesive, etc. In some instances, a hydrophilic coating 340 may be applied over all or some portion of the length of the second polymer coating 328. In some aspects, the hydrophilic coating 340 may not be applied to the proximal connector, i.e. at the proximal portion, the hydrophilic coating 340 starts just distal of the conductive portions 132. In some instances, the hydrophilic coating 340 may be provided between the second polymer coating 328 and the conductive portions 132, 134, and 136. In that regard, an ablation goes through the hydrophilic coating 340 and the second polymer coating 328 to establish electrical communication between respective ones of the conductive portions 132, 134, and 136 and respective ones of the conductive member 324.

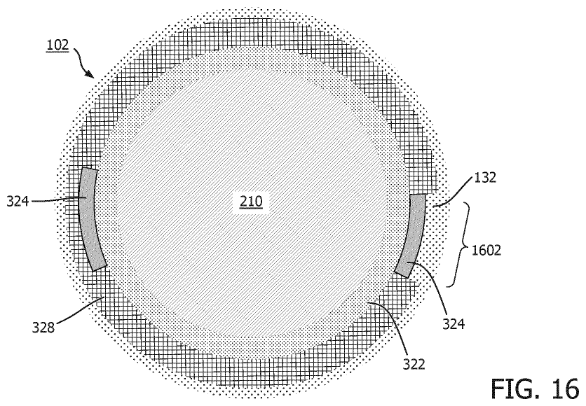

FIG. 16 illustrates a cross-sectional view of an intravascular device 102 of FIG. 15, as seen along the lines of the section B-B taken therein, according to aspects of the present disclosure. In the aspect of FIG. 16, the first polymer coating 322 may be applied along a length of the intravascular device 102 from a proximal end of the proximal core 220 to a distal end of the distal core 210 surrounding the proximal core 220 and the distal core 210. In some aspects, one or more conductive layers 324 may then be applied along the length of the intravascular device 102 on top of the first polymer coating 322 from the proximal end of the proximal core 220 to the distal end of the distal core 210. In some aspects, the second polymer coating 328 may be applied along a length of the intravascular device 102 from a proximal end of the proximal core 220 to a distal end of the distal core 210 surrounding the proximal core 220 and the distal core 210. Prior to applying the conductive portion 132 to a proximal end of the proximal core 220, a portion 1602 of the second polymer coating 328 may be ablated exposing the conductive layers 324. The conductive portion 132 may then be applied to a proximal end of the proximal core 220 such that the conductive portion 132 is electrically and mechanically coupled to and in electrical communication with the conductive layers 324 by, e.g., soldering, ultrasonic welding, conductive adhesive, etc.

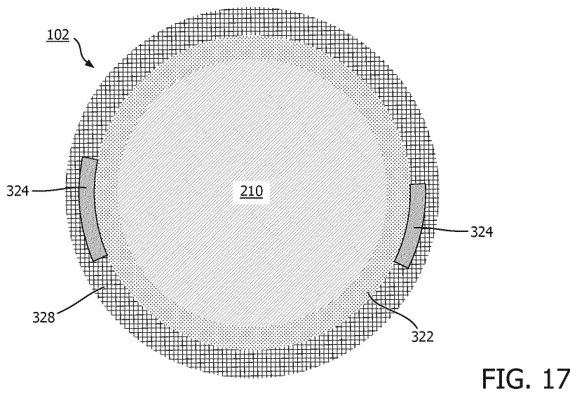

FIG. 17 illustrates a cross-sectional view of an intravascular device 102 of FIG. 15, as seen along the lines of the section C-C taken therein, according to aspects of the present disclosure. In the aspect of FIG. 17, the first polymer coating 322 may be applied along a length of the intravascular device 102 from a proximal end of the proximal core 220 to a distal end of the distal core 210 surrounding the proximal core 220 and the distal core 210. In some aspects, one or more conductive layers 324 may then be applied along the length of the intravascular device 102 on top of the first polymer coating 322 from the proximal end of the proximal core 220 to the distal end of the distal core 210. In some aspects, the second polymer coating 328 may be applied along a length of the intravascular device 102 from a proximal end of the proximal core 220 to a distal end of the distal core 210 surrounding the one or more conductive layers 324 and the first polymer coating 322.

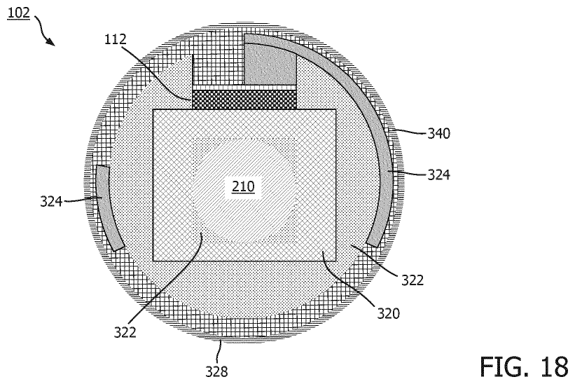

FIG. 18 illustrates a cross-sectional view of an intravascular device 102 of FIG. 11, as seen along the lines of the section A-A taken therein, according to aspects of the present disclosure. FIG. 18 includes features similar to those described in FIG. 12. In addition to those elements shown in FIG. 12, FIG. 18 further depicts the second polymer coating 328 that may be applied along a length of the intravascular device 102 from a proximal end of the proximal core 220 to a distal end of the distal core 210 surrounding the proximal core 220 and the distal core 210. In some instances, a hydrophilic coating 340 may be applied over all or some portion of the length of the second polymer coating 328. In some aspects, the hydrophilic coating 340 may not be applied to the sensor 112, i.e. at the distal portion, the hydrophilic coating 340 starts just proximal of the sensor 112.

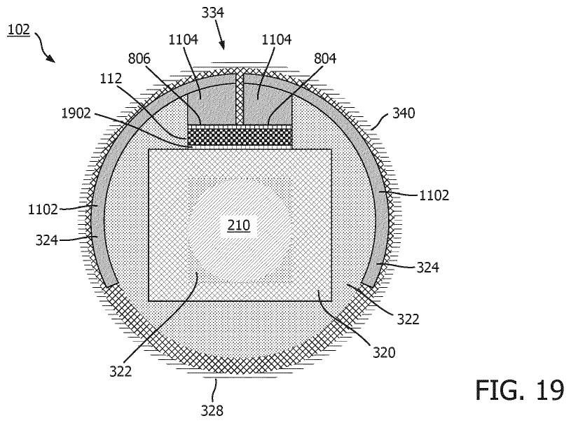

FIG. 19 illustrates a cross-sectional view of an intravascular device 102 according to aspects of the present disclosure. In the aspect of FIG. 19, the sensor mount/housing 320 is coupled to the distal end of the distal core 210. The first polymer coating 322 may then be applied to the distal core 210 and the sensor mount/housing 320 surrounding the distal core 210 and the sensor mount/housing 320 and filling any gaps between the distal core 210 and the sensor mount/housing 320. The one or more portions 334 of the first polymer coating 322 is ablated and/or ground to form a sensor shape so that a sensor 112 may be coupled to the sensor mount/housing 320. One or more conductive layers 324 may then be applied to the intravascular device 102 on top of the first polymer coating 322. In some instance, the one or more conductive layers 324 partially wrap around a diameter of the distal core 210 such that a first end 1102 of the partial wrap of the one or more conductive layers 324 couples to the one or more conductive layers 324 applied along the length of the intravascular device 102 and a second end 1104 of the partial wrap couples to electrical contact pads 804 or 806, respectively, of the sensor 112. In some instances, sensor 112 may include a conductive bottom 1902 that may be mechanically and electrically coupled to the sensor mount/housing 320. In some instances, all or part of the sensor mount housing 320 may be conductive so as to couple to the conductive material within the body of the sensor 112 thereby providing a conductive pathway. In such instances, an electrical pathway is provided between the sensor 112 and the conductive portion 132, 134, or 136: the sensor 112 to the sensor mount/housing 320 to the distal core 210 to the proximal core 220 to the conductive portion 132, 134, or 136 (and vice versa). In some instances, sensor 112 may include a conductive bottom 1902 that may be mechanically and electrically coupled to the distal core 210. For example, a portion of the sensor mount/housing 320 may be omitted between conductive bottom 1902 and the distal core 210 so that the conductive bottom 1902 directly contacts the distal core 210, such that the sensor 112 is directly mechanically and electrically coupled to the distal core 210. In such instances, an electrical pathway is provided between the sensor 112 and the conductive portion 132, 134, or 136: the sensor 112 to the distal core 210 to the proximal core 220 to the conductive portion 132, 134, or 136 (and vice versa). In such instances, the sensor mount/housing 320 may be non-conductive.

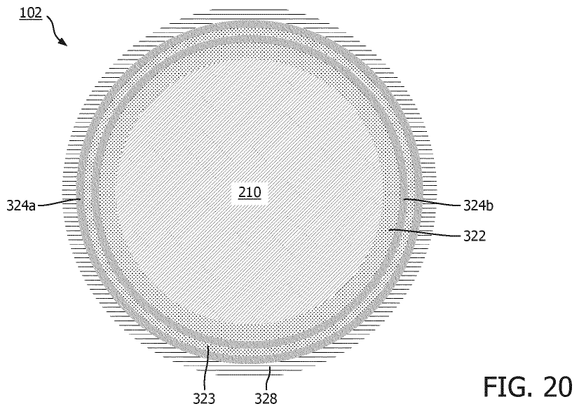

FIG. 20 illustrates a cross-sectional view of an intravascular device 102 according to aspects of the present disclosure. In the aspect of FIG. 17, the first polymer coating 322 may be applied along a length of the intravascular device 102 from a proximal end of the proximal core 220 to a distal end of the distal core 210 surrounding the proximal core 220 and the distal core 210. In some aspects, a first conductive layer 324a may then be applied along the length of the intravascular device 102 on top of the first polymer coating 322 from the proximal end of the proximal core 220 to the distal end of the distal core 210. In some aspects, the second polymer coating 323 may be applied along a length of the intravascular device 102 from a proximal end of the proximal core 220 to a distal end of the distal core 210 surrounding the first conductive layer 324a. In some aspects, a second conductive layer 324b may then be applied along the length of the intravascular device 102 on top of the second polymer coating 323 from the proximal end of the proximal core 220 to the distal end of the distal core 210. In some instances, the shape of the first conductive layer 324a and the second conductive layer 324b may be completely annular or partially annular. In some instances, the second polymer coating 323 between the first conductive layer 324a and the second conductive layer 324b provides electrical isolation between the two conductive layers. In some aspects, the third polymer coating 328 may be applied along a length of the intravascular device 102 from a proximal end of the proximal core 220 to a distal end of the distal core 210 surrounding the second conductive layer 324b. In some instances, the first conductive layer 324a and the second conductive layer 324b provide two electrical pathways between the sensor 112 and two of the conductive portions 132, 134, or 136. In such instances, the distal core 210 and the proximal core 220, which can be electrically and mechanically coupled to each other, provide a third electrical pathway between the sensor 112 and the third of the conductive portions 132, 134, or 136.

Accordingly, it may be seen that the electrical trace assembly advantageously enables the use of conductive materials that may be applied in a thinner layer reducing the dimensions (thickness) of the electrical connection, facilitating centering of the distal core within the wire body and removing irregularities in the surface. The sensor is electrically connected using a bonding process and additional bonding materials. Ink (3D) traces can be directly connected to the sensor or sensor mount, without the need for additional bonding process that require additional forces on the sensor. A straightness and torque response of the intraluminal device may also be improved because ribbons and/or multi-filar conductor bundles, which would be reduced or eliminated, add stiffness and local torques to a guidewire or catheter. A number of variations are possible on the examples and embodiments described above.

The logical operations making up the aspect of the technology described herein are referred to variously as operations, steps, objects, elements, components, or modules. Furthermore, it should be understood that these may be arranged or performed in any order, unless explicitly claimed otherwise or a specific order is inherently necessitated by the claim language. It should further be understood that the described technology may be employed in single-use and multi-use electrical and electronic devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the metal ink conductor assembly. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the metal ink conductor assembly as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. An apparatus, comprising:
an intravascular guidewire comprising:
a flexible elongate member configured to be positioned within a blood vessel of a patient, wherein the flexible elongate member comprises a proximal portion and a distal portion, wherein the proximal portion comprises a proximal core wire and the distal portion comprises a distal core wire;
a sensor disposed at the distal portion of the flexible elongate member, wherein the sensor is configured to obtain medical data related to the blood vessel while the flexible elongate member is positioned within the blood vessel;
a connector disposed at the proximal portion of the flexible elongate member; and
a conductive member establishing electrical communication between the sensor and the connector, wherein the conductive member is continuous between sensor and the connector.

2. The apparatus of claim 1, wherein a distal portion of the conductive member is directly coupled to the sensor.

3. The apparatus of claim 1,
wherein the connector comprises a conductive band,
wherein a proximal portion of the conductive member is directly coupled to the conductive band.

4. The apparatus of claim 1, wherein the intravascular guidewire comprises plurality of conductive members establish electrical communication between the sensor and the connector, wherein each conductive member of the plurality of conductive members is continuous between the sensor and the connector.

5. The apparatus of claim 1,
wherein a distal portion of the proximal core wire is coupled to a proximal portion of the distal core wire at a location along a length of the flexible elongate member,
wherein the conductive member is continuous across the location.

6. The apparatus of claim 5,
wherein the location comprises a tubular member,
wherein the distal portion of the proximal core wire and the proximal portion of the distal core wire are disposed inside of the tubular member,
wherein the conductive member is disposed outside of the tubular member.

7. The apparatus of claim 1,
wherein the flexible elongate member further comprises a first polymer layer disposed over the proximal core wire and the distal core wire,
wherein the first polymer layer is configured to provide insulation for the conductive member from the proximal core wire and the distal core.

8. The apparatus of claim 7,
wherein the intravascular guidewire further comprises a sensor mount,
wherein the sensor is coupled to the sensor mount,
wherein the first polymer layer is disposed over the sensor mount.

9. The apparatus of claim 8, wherein the first polymer layer comprises an opening exposing the sensor.

10. The apparatus of claim 7, wherein the flexible elongate member further comprises a second polymer layer disposed over the conductive member.

11. The apparatus of claim 10, wherein the flexible elongate member further comprises a hydrophilic coating disposed over the second polymer layer.

12. The apparatus of claim 10, wherein, in a cross-section, a perimeter of the conductive member is completely surrounded by at least one of the first polymer layer or the second polymer layer.

13. The apparatus of claim 1, wherein the conductive member comprises an electrical trace.

14. The apparatus of claim 11, wherein the electrical trace comprises at least one of a conductive ink, metal disposition, electroplating, three-dimensional printed conductive polymer or metal, or extruded conductive material.

15. The apparatus of claim 1, wherein the sensor comprises at least one of a pressure sensor or a flow sensor.

16. An apparatus, comprising:
an intravascular guidewire comprising:
a flexible elongate member configured to be positioned within a blood vessel of a patient, wherein the flexible elongate member comprises a proximal portion and a distal portion, wherein the proximal portion comprises a proximal core wire and the distal portion comprises a distal core wire, wherein proximal core wire is coupled to the distal core wire at a location along a length of the flexible elongate member;
at least one of a pressure sensor or a flow sensor disposed at the distal portion of the flexible elongate member, wherein at least one of the pressure sensor or the flow sensor is configured to obtain at least one of pressure data or flow data, respectively, related to the blood vessel while the flexible elongate member is positioned within the blood vessel;
a connector disposed at the proximal portion of the flexible elongate member; and
a conductive member establishing electrical communication between the sensor and the connector, wherein the conductive member is continuous between sensor and the connector, including across the location.
